(19) United States
(12) Patent Application Publication
(10) Pub. No.: US 2025/0155252 A1
(43) Pub. Date: May 15, 2025

(54) STOPOVER RECOMMENDATION METHOD BASED ON FATIGUE STATUS AND NAVIGATION SYSTEM FOR PERFORMING THE SAME

(71) Applicant: HYUNDAI AUTOEVER CORP., Seoul (KR)

(72) Inventors: Moon Jeong KIM, Seoul (KR); Won Mi AHN, Seoul (KR); Yeon Su KIM, Seoul (KR); Suk Ho LEE, Seoul (KR); So Jung KIM, Seoul (KR); Hwa Hyeon HONG, Seoul (KR)

(21) Appl. No.: 18/942,047

(22) Filed: Nov. 8, 2024

(30) Foreign Application Priority Data
Nov. 9, 2023 (KR) 10-2023-0154237

(51) Int. Cl. G01C 21/34; A61B 5/00; A61B 5/024; A61B 5/16; A61B 5/18; G07C 5/02

(52) U.S. Cl. CPC G01C 21/3484; A61B 5/024; A61B 5/165; A61B 5/18; A61B 5/7264; G01C 21/3476; G07C 5/02; A61B 2503/22

(57) ABSTRACT

Stopover recommendation methods and devices are described. According to one embodiment, a method comprises acquiring vehicle information indicating a state of a vehicle and user information about a user, detecting a fatigue status of the user using the vehicle information and the user information, classifying the fatigue status into a drowsiness-inducing fatigue, a negative emotion fatigue, or a lack-of-exercise fatigue, wherein the negative emotion fatigue includes a relaxed fatigue and a tense fatigue, recommending a stopover based on the fatigue status of the user and a past destination setting history of the user, displaying detailed information on the recommended stopover, and determining a route including the recommended stopover as a final route in response to an approval of the user on the recommended stopover.

### CROSS-REFERENCE TO RELATED APPLICATION

This application claims priority from Korean Patent Application No. 10-2023-0154237 filed on Nov. 9, 2023 in the Korean Intellectual Property Office, and all the benefits accruing therefrom under 35 U.S.C. 119, the contents of which in its entirety are herein incorporated by reference.

### BACKGROUND

### Field

The present disclosure relates to a stopover recommendation method based on a fatigue status, and a navigation system for performing the same. More specifically, the present disclosure relates to a method for detecting a fatigue status based on vehicle information indicating a state of a vehicle or user information, classifying the detected fatigue status based on a fatigue type, and recommending a stopover appropriate for each fatigue type, and a navigation system for performing the same.

### Description of Related Art

Generally, each of various mobility apparatuses employs a navigation system equipped with a GPS (Global Positioning System) to identify a current location and a current moving speed thereof or to determine a moving speed thereof. The navigation system receives a radio wave indicating a latitude, a longitude, an altitude, etc. from a plurality of artificial satellites, calculates a current location of a moving object to identify the current location of the moving object, and identifies a destination input by a user, and determines a moving route starting from the current location and arriving at the destination.

The navigation system determines the route based on various information related to driving, such as to a distance the destination, an arrival time, a cost such as an optimal distance to the destination, the shortest distance, a traffic condition, use of free or toll road, etc. The navigation system includes not only a component for receiving the destination directly from the user, but also a component that searches for the current location or the surroundings around the destination, and a component that recommends a stopover point.

The navigation system does not simply provide the shortest route for the user to reach the destination. Further, it is important to recommend an appropriate stopover for the user's rest when the user is tired. In particular, instead of recommending a general stopover when the user is tired, a detected fatigue status needs to be classified based on the fatigue type, and an individualized stopover need to be recommended according to each of different fatigue types.

### SUMMARY

A technical purpose to be achieved using embodiments of the present disclosure is to provide a method for detecting a fatigue status based on vehicle information indicating a state of a vehicle or user information, classifying the detected fatigue status based on a fatigue type, and recommending a rest-purposed stopover appropriate for each fatigue type, and a navigation system for performing the same.

The technical purposes of the present disclosure are not limited to the technical purposes mentioned above, and other technical purposes not mentioned may be clearly understood by those skilled in the art from descriptions as set forth below.

According to an aspect of the present disclosure, a stopover recommendation method may be performed by a computing device, and the method may comprise: acquiring vehicle information indicating a state of a vehicle and user information about a user; detecting a fatigue status of the user using the acquired vehicle information and user information; classifying the detected fatigue status into one of drowsiness-inducing fatigue, negative emotion fatigue, and lack-of-exercise fatigue, wherein the negative emotion fatigue includes relaxed fatigue and tense fatigue; recommending a stopover based on the fatigue classification result and the user's past destination setting history; displaying detailed information on the recommended stopover; and determining a route including the recommended stopover as a final route, in response to the user's approval with the recommended stopover.

In one embodiment, the vehicle information may include at least one of information on a location (GPS) of a vehicle, a driving speed, a total driving time duration, a window opening time or a frequency of window opening and closing, a brake pedal stepping strength, a signal indicating a departure of a preceding vehicle, information on media being played, a frequency of air conditioner control, whether ADAS (Advanced Driver Assistance Systems function) is used, and whether a seat massage function is used, wherein the user information may be extracted from usage history of the computing device and the user device of the user, wherein the user information may include at least one of the user's heart rate, a volume of voice during a call, the user's past destination setting history, whether or not the user has visited a specific location, and the user's posture.

In one embodiment, the classifying of the detected fatigue status may include: when a driving timing is evening, night, or dawn, when a total driving time duration is equal to or greater than a preset first threshold time duration, or when a window opening time duration is equal to or greater than a preset second threshold time duration, classifying the detected fatigue status as the drowsiness-inducing fatigue, wherein each of the first threshold time duration and the second threshold time duration may be determined based on a driving pattern of the user acquired from the vehicle.

In one embodiment, the classifying of the detected fatigue status may include: when the heart rate is smaller than a reference value, when a brake pedal stepping strength is smaller than an average brake pedal stepping strength of the user, when a difference between a speed limit on a road on which a vehicle is currently driving and the current driving speed is larger than a predetermined threshold value, or when the vehicle does not start despite a preceding vehicle movement-start notification signal, classifying the detected fatigue status as the relaxed fatigue of the negative emotion fatigue, wherein the brake pedal stepping strength may be stored in the vehicle every time the user drives the vehicle, wherein the average brake pedal stepping strength may be calculated from values of the brake pedal stepping strength stored in the vehicle, wherein the predetermined threshold value may be determined as an average of difference values between a driving speed stored in the navigation system or the vehicle and the speed limit on the road on which the vehicle is currently driving.

In one embodiment, the classifying of the detected fatigue status may include: when the heart rate is higher than a reference value, or when the volume of the call voice is greater than an average volume of the user's call voice, classifying the detected fatigue status as the tense fatigue of the negative emotion fatigue, wherein the average volume of the user's call voice may be calculated from the user's call record stored in the user device.

In one embodiment, the classifying of the detected fatigue status may include: when the user's posture is different from a correct posture, or when the seat massage function is activated, classifying the detected fatigue status as the lack-of-exercise fatigue.

In one embodiment, the recommending of the stopover may include: determining whether the user's fatigue status is classified as the drowsiness-inducing fatigue, when the user's fatigue status is classified as the drowsiness-inducing fatigue, determining whether the current time is night or dawn and the driving time duration has exceeded a predetermined first threshold time duration, when the current time is night or dawn and the total driving time duration has exceeded the predetermined first threshold time duration, recommending a nearest accommodation place among searched accommodation places based on a current location as the stopover, when the current time is not night or dawn, or when the total driving time duration does not exceed the first threshold time duration, recommending a nearest drowsiness shelter or rest area searched based on the current location as the stopover, wherein the first threshold time duration may be determined based on the user's driving pattern acquired from the vehicle.

In one embodiment, the recommending of the stopover may include: determining whether the user's fatigue status is classified as the negative emotion fatigue, when the user's fatigue status is classified as the negative emotion fatigue, searching for a destination that the user visited on a holiday, based on the user's past destination setting history; and recommending a park or a cafe among the searched destinations as the stopover.

In one embodiment, the recommending of the stopover may include: determining whether the user's fatigue status is classified as the lack-of-exercise fatigue; when the user's fatigue status is classified as the lack-of-exercise fatigue, determining whether there is an exercise place that the user visited, based on the user's past destination setting history; and when there is the exercise place that the user visited, recommending the exercise place that the user visited as the stopover; and when there is no exercise place that the user visited, recommending a place at which the vehicle can stop and park as the stopover.

In one embodiment, wherein the detailed information on the recommended stopover may include the user's fatigue status based on which the stopover is recommended, a type of rest that may be taken in the stopover, and an expected driving time to drive along a route including the recommended stopover thereto.

According to another aspect of the present disclosure, a computing device may comprise: a processor; and a memory connected to the process and configured to store therein instructions, wherein when the instructions are executed by the processor, the instructions may cause the processor to: acquire vehicle information indicating a state of a vehicle and user information about a user; detect a fatigue status of the user using the acquired vehicle information and user information; classify the detected fatigue status into one of drowsiness-inducing fatigue, negative emotion fatigue, and lack-of-exercise fatigue, wherein the negative emotion fatigue includes relaxed fatigue and tense fatigue; recommend a stopover based on the fatigue classification result and the user's past destination setting history; display detailed information on the recommended stopover; and determine a route including the recommended stopover as a final route, in response to the user's approval with the recommended stopover.

In one embodiment, the vehicle information may include at least one of information on a location (GPS) of a vehicle, a driving speed, a total driving time duration, a window opening time or a frequency of window opening and closing, a brake pedal stepping strength, a signal indicating a departure of a preceding vehicle, information on media being played, a frequency of air conditioner control, whether ADAS (Advanced Driver Assistance Systems function) is used, and whether a seat massage function is used, wherein the user information may be extracted from usage history of the computing device and the user device of the user, wherein the user information may include at least one of the user's heart rate, a volume of voice during a call, the user's past destination setting history, whether or not the user has visited a specific location, and the user's posture.

In one embodiment, the classifying of the detected fatigue status may include: when a driving timing is evening, night, or dawn, when a total driving time duration is equal to or greater than a preset first threshold time duration, or when a window opening time duration is equal to or greater than a preset second threshold time duration, classifying the detected fatigue status as the drowsiness-inducing fatigue, wherein each of the first threshold time duration and the second threshold time duration may be determined based on a driving pattern of the user acquired from the vehicle.

In one embodiment, the classifying of the detected fatigue status may include: when the heart rate is smaller than a reference value, when a brake pedal stepping strength is smaller than an average brake pedal stepping strength of the user, when a difference between a speed limit on a road on which a vehicle is currently driving and the current driving speed is larger than a predetermined threshold value, or when the vehicle does not start despite a preceding vehicle movement-start notification signal, classifying the detected fatigue status as the relaxed fatigue of the negative emotion fatigue, wherein the brake pedal stepping strength may be stored in the vehicle every time the user drives the vehicle, wherein the average brake pedal stepping strength may be calculated from values of the brake pedal stepping strength stored in the vehicle, wherein the predetermined threshold value may be determined as an average of difference values between a driving speed stored in the navigation system or the vehicle and the speed limit on the road on which the vehicle is currently driving.

In one embodiment, the classifying of the detected fatigue status may include: when the heart rate is higher than a reference value, or when the volume of the call voice is greater than an average volume of the user's call voice, classifying the detected fatigue status as the tense fatigue of the negative emotion fatigue, wherein the average volume of the user's call voice may be calculated from the user's call record stored in the user device.

In one embodiment, the classifying of the detected fatigue status may include: when the user's posture is different from a correct posture, or when the seat massage function is activated, classifying the detected fatigue status as the lack-of-exercise fatigue.

In one embodiment, the recommending of the stopover may include: determining whether the user's fatigue status is classified as the drowsiness-inducing fatigue, when the user's fatigue status is classified as the drowsiness-inducing fatigue, determining whether the current time is night or dawn and the driving time duration has exceeded a predetermined first threshold time duration, when the current time is night or dawn and the total driving time duration has exceeded the predetermined first threshold time duration, recommending a nearest accommodation place among searched accommodation places based on a current location as the stopover, when the current time is not night or dawn, or when the total driving time duration does not exceed the first threshold time duration, recommending a nearest drowsiness shelter or rest area searched based on the current location as the stopover, wherein the first threshold time duration may be determined based on the user's driving pattern acquired from the vehicle.

In one embodiment, the recommending of the stopover may include: determining whether the user's fatigue status is classified as the negative emotion fatigue, when the user's fatigue status is classified as the negative emotion fatigue, searching for a destination that the user visited on a holiday, based on the user's past destination setting history; and recommending a park or a cafe among the searched destinations as the stopover.

In one embodiment, the recommending of the stopover may include: determining whether the user's fatigue status is classified as the lack-of-exercise fatigue; when the user's fatigue status is classified as the lack-of-exercise fatigue, determining whether there is an exercise place that the user visited, based on the user's past destination setting history; and when there is the exercise place that the user visited, recommending the exercise place that the user visited as the stopover; and when there is no exercise place that the user visited, recommending a place at which the vehicle can stop and park as the stopover.

In one embodiment, the detailed information on the recommended stopover may include the user's fatigue status based on which the stopover is recommended, a type of rest that may be taken in the stopover, and an expected driving time to drive along a route including the recommended stopover thereto.

### BRIEF DESCRIPTION OF DRAWINGS

The above and other aspects and features of the present disclosure will become more apparent by describing in detail illustrative embodiments thereof with reference to the attached drawings, in which:

FIG. 1 shows an example of an environment to which a navigation system for performing a stopover recommendation method based on a fatigue status according to an embodiment of the present disclosure;

FIG. 2 illustrates examples of types of a fatigue status classified according to an embodiment of the present disclosure;

FIG. 3 illustrates examples of fatigue type-related fatigue detection factors according to an embodiment of the present disclosure.

FIG. 4 illustrates an example of an initial screen of the navigation system of FIG. 1;

FIG. 5 illustrates an example of a route guidance screen of the navigation system of FIG. 1;

FIG. 6 is a flowchart for illustrating a stopover recommendation method based on a fatigue status according to an embodiment of the present disclosure;

FIGS. 7 to 9 are flowcharts for illustrating an embodiment of an operation of recommending a stopover based on a fatigue classification result of FIG. 6 and a past destination setting history;

FIG. 10 illustrates examples of stopovers that may be recommended based on fatigue types according to an embodiment of the present disclosure; and

FIG. 11 is a block diagram showing a hardware configuration of a computing device for performing a stopover recommendation method based on a fatigue status according to an embodiment of the present disclosure.

### DETAILED DESCRIPTIONS

Preferred embodiments of the present disclosure will hereinafter be described in detail with reference to the accompanying drawings. The advantages, features, and methods of achieving them of the present disclosure will become clearer with the embodiments described in detail along with the accompanying drawings. However, the present disclosure is not limited to the embodiments described below and can be implemented in various different forms. These embodiments are provided only to make the disclosure complete and fully inform those of ordinary skill in the technical field to which the present disclosure belongs, and the present disclosure is defined only by the scope of the claims.

It is noted that the same reference numerals are used for the same elements across different drawings as far as possible. Furthermore, in describing the present disclosure, detailed descriptions of known configurations or functions will be omitted when they may obscure the essence of the present disclosure.

Unless defined otherwise, all terms used herein (including technical and scientific terms) can have the meaning commonly understood by one of ordinary skill in the art to which the present disclosure belongs. Terms defined in commonly used dictionaries are not interpreted in an ideal or excessive manner unless explicitly defined otherwise. The terms used in the present specification are for the purpose of describing particular embodiments only and are not intended to limit the invention. In this specification, the singular forms include plural forms unless the context clearly indicates otherwise.

Furthermore, in describing the components of the present disclosure, terms such as first, second, A, B, (a), (b), etc., may be used. These terms are intended to distinguish the components from others, and the essence, order, or sequence of such components is not limited by these terms. If a component is stated as being “connected,” “coupled,” or “linked” to another component, the component can be directly connected or linked to the other component, but it should be understood that there may also exist other components “connected,” “coupled,” or “linked between them.

The terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

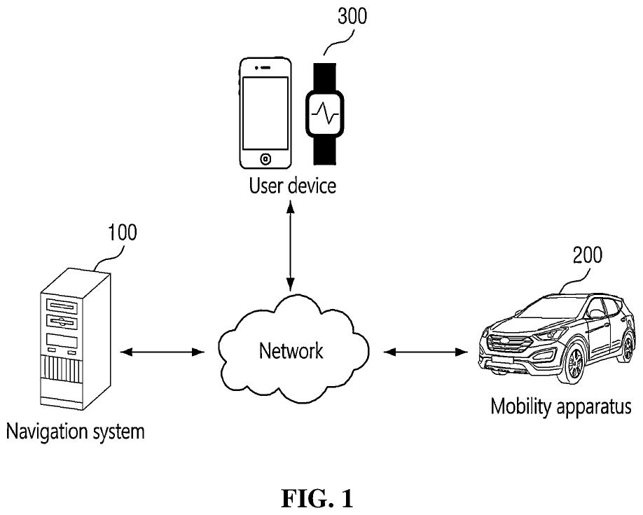

FIG. 1 shows an example of an environment to which a navigation system 100 for performing a stopover recommendation method based on a fatigue status according to an embodiment of the present disclosure may be applied. The navigation system 100 may acquire vehicle information indicating a state of the vehicle and user information through communication with the mobility apparatus 200 and the user device 300, and may detect the user's fatigue status based on the acquired information. In addition, the detected fatigue status may be classified based on the fatigue type by the navigation system 100. A stopover for rest and fatigue relief may be recommended according to the type of the fatigue status by the navigation system 100. For the convenience of the following descriptions, the mobility apparatus 200 is assumed to be the vehicle, and in this case, the mobility apparatus 200 and the vehicle 200 will be referred to by the same reference number. However, the present disclosure is not limited thereto, and the mobility apparatus 200 may be embodied as transportation means other than the vehicle.

For example, the information indicating the state of the vehicle 200 may include information on a location (GPS) of a vehicle, a driving speed, a total driving time duration, a window opening time or a frequency of window opening and closing, a brake pedal stepping strength, a signal indicating a departure of a preceding vehicle, information on media being played, a frequency of air conditioner control, whether ADAS (Advanced Driver Assistance Systems function) is used, whether a seat massage function is used, etc. Furthermore, the user information may include the user's heart rate, a volume of voice during a call, the user's past destination setting history, whether or not he or she has visited a specific location, etc., which may be extracted from the navigation system 100 and the user device 300. Furthermore, the user information may further include the user's posture, a frequency of changing the posture correctly, a frequency of yawning, etc., which may be extracted from a camera installed in the vehicle 200.

The navigation system 100 according to an embodiment of the present disclosure may detect the user's fatigue status based on the vehicle information and the user information. For example, the user's fatigue status may be detected when the total driving time duration has exceeded a threshold time duration, when yawning occurs frequently, when a difference between a speed limit and a current driving speed is gradually increasing (i.e., driving at an increasingly lower speed), when the vehicle does not start immediately despite a preceding vehicle movement-start notification signal, when the heart rate is low or high, when the air conditioner is frequently turned on or off, when the posture is constantly corrected, or when the ADAS function is used for an excessively long time, etc.

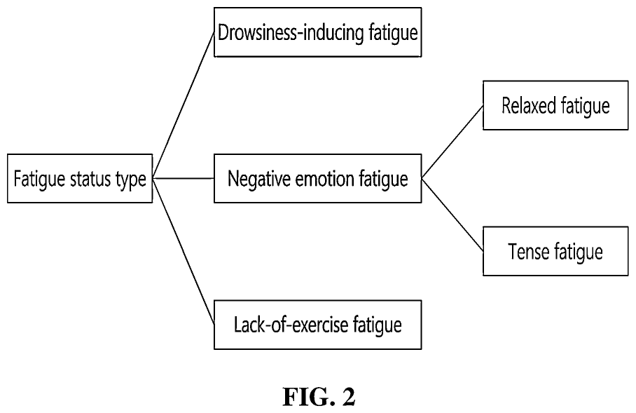

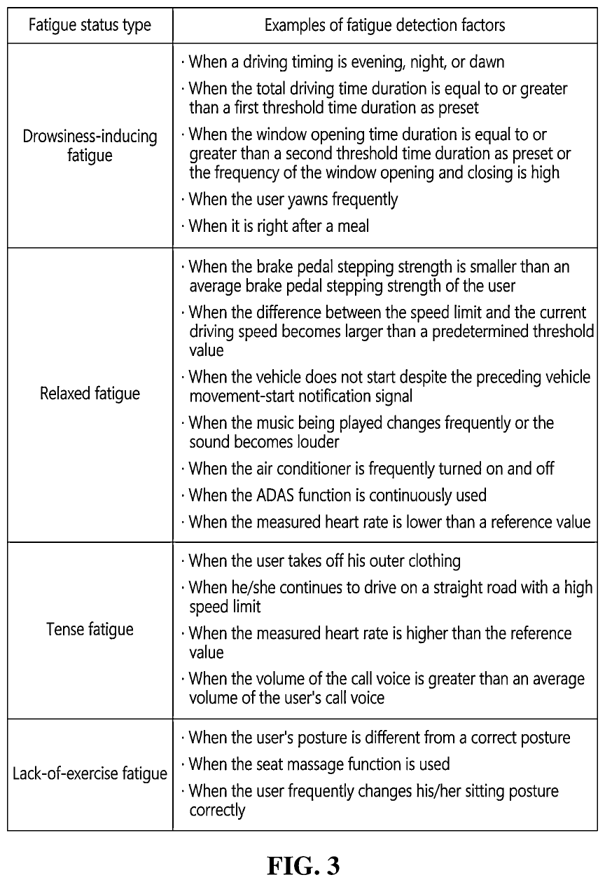

Furthermore, the navigation system 100 according to an embodiment of the present disclosure may classify the detected fatigue statuses based on a fatigue type. For example, the fatigue statuses may be classified into drowsiness-inducing fatigue, negative emotion fatigue, and lack-of-exercise fatigue. That is, the fatigue type may include into the drowsiness-inducing fatigue, negative emotion fatigue, and lack-of-exercise fatigue. The negative emotions may be classified into relaxed fatigue and tense fatigue. Thereafter, the navigation system 100 may recommend an appropriate stopover based on the classified type of the fatigue status. For example, when the user is in the drowsiness-inducing fatigue status, a drowsiness shelter or a rest area for a short rest may be recommended as a stopover, or in some cases, a place where the user may stay may be recommended as a stopover. When the user is in the relaxed or tense fatigue status, a cafe or park for emotional relaxation may be recommended as a stopover, or it may be more efficient that a place that the user usually visits for relaxation is recommended. When the user is in a lack-of-exercise fatigue status, and when there is a place where the user regularly visits for exercise, that place may be recommended as a stopover. Alternatively, a place where the user may stop for simple stretching, etc. may be recommended as a stopover. This will be described in more detail with reference to FIG. 2 and FIG. 3.

The navigation system 100 may display detailed information about the stopover along with the stopover recommendation. The detailed information may include the user's fatigue status based on which the stopover is recommended, a type of rest that may be taken in the stopover, and an expected driving time to drive along a route including the recommended stopover thereto. After checking the user's fatigue status, the recommended stopover, and the related detailed information, the user of the vehicle may determine the route including the stopover as a final route. In one example, the user may determine an existing route toward the destination as the final route regardless of the stopover recommendation result.

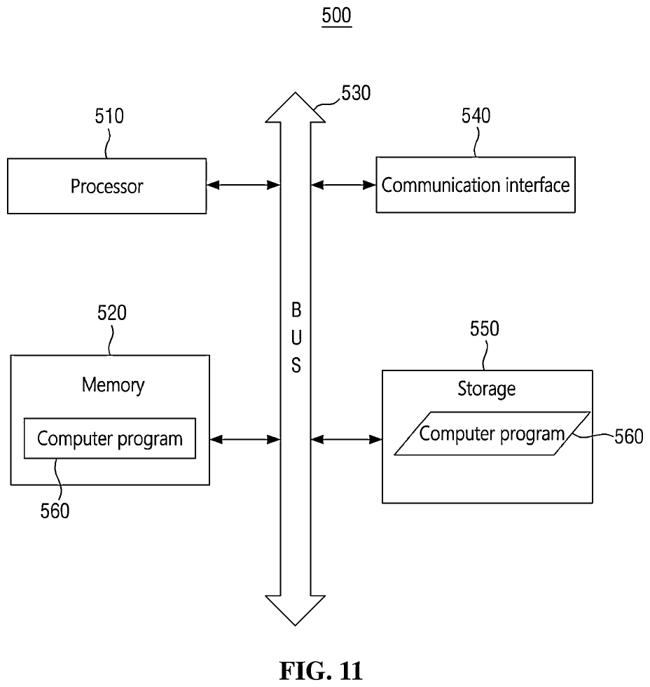

The navigation system 100 according to some embodiments of the present disclosure may be implemented on at least one computing device. For example, all functions of the navigation system 100 may be implemented on one computing device. Alternatively, some functions of the navigation system 100 may be implemented on a first computing device, and the remaining functions thereof may be implemented on a second computing device. Alternative, a specific function of the navigation system 100 may be implemented on a plurality computing devices. For example, a stopover recommendation function of the navigation system 100 and a function of displaying information about the destination thereof may be implemented separately on different computing devices. The computing device may include any device equipped with a computing means and a communication means. An embodiment in which the navigation system 100 is implemented as a computing device is described in more detail with reference to FIG. 11.

The user device 300 may be any personal computing device used by the user of the vehicle 200. For example, the user device 300 may include a smart phone, a smart watch, a tablet, a desktop, a laptop, etc. However, the present disclosure is not limited thereto, and the user device 300 may be embodied as any computing device equipped with a computing means and a communication means. As described above, the navigation system 100 may communicate with the user device 300 and may acquire and use usage history of the user device 300, biometric information measured from the user device 300, etc. as the user information.

Furthermore, the components illustrated in FIG. 1 may communicate with each other through a network. For example, the network may be embodied as any type of a wired/wireless network such as a local area network (LAN), a wide area network (WAN), a mobile radio communication network, and Wibro (Wireless Broadband Internet).

FIG. 2 shows an example of the types of the fatigue statuses as classified according to an embodiment of the present disclosure.

Referring to FIG. 2, the fatigue type may be one of the drowsiness-inducing fatigue, the negative emotion fatigue, and the lack-of-exercise fatigue. The negative emotion fatigue may be one of the relaxed fatigue and the tense fatigue. When the user is in the drowsiness-inducing fatigue status which is maintained continuously, this may induce drowsiness driving. Thus, it is a state that requires immediate rest of the user. When the user is in the negative emotion fatigue status, this indicates that the user is in an excessive relaxed state or tense state. Thus, it is a state that requires emotional stability for safe driving. When the user is in the lack-of-exercise fatigue status which is maintained continuously, this may have a negative effect on the user's health or posture. Thus, it is a state that requires exercise. However, the classification of the fatigue types as shown in FIG. 2 is merely an example, and the present disclosure is not limited thereto, and the fatigue statuses may be classified into various fatigue types other than those shown in FIG. 2.

FIG. 3 shows an example of the fatigue type-related fatigue detection factors according to an embodiment of the present disclosure. Referring to FIG. 3, it shows which factor may cause the detected fatigue status to be classified into one of the drowsiness-inducing fatigue, the relaxed fatigue, the tense fatigue, and the lack-of-exercise fatigue. As described with reference to FIG. 1, the fatigue status may be detected based on the vehicle information or the user information.

First, when a driving timing is evening, night, or dawn, when the total driving time duration is equal to or greater than a first threshold time duration as preset, when the window opening time duration is equal to or greater than a second threshold time duration as preset or the frequency of the window opening and closing is high, when the user yawns frequently, or when it is right after a meal (for example, when a current time is in a meal timing zone and a departure location is a restaurant), the user's fatigue status may be classified as the drowsiness-inducing fatigue status.

For example, each of the first threshold time duration and the second threshold time duration described above may be determined based on the user's driving pattern acquired from the vehicle.

Next, when the brake pedal stepping strength is smaller than an average brake pedal stepping strength of the user, when the difference between the speed limit and the current driving speed becomes larger than a predetermined threshold value (i.e., driving at a slower speed than the speed limit), when the vehicle does not start despite the preceding vehicle movement-start notification signal, when the music being played changes frequently or the sound becomes louder, when the air conditioner is frequently turned on and off, when the ADAS function is continuously used, or when the measured heart rate is lower than a reference value, the user's fatigue status may be classified as the relaxed fatigue status among the negative emotion fatigues. For example, the brake pedal stepping strength may be stored in the vehicle every time the user drives, and the above-described average brake pedal stepping strength may be calculated from the values stored in the vehicle. Furthermore, the predetermined threshold value related to the difference between the speed limit and the current driving speed as described above may be determined as an average of difference values between the driving speed and the speed limit on a specific road as stored in the navigation system 100 or the vehicle.

In particular, based on a value of the difference between the speed limit and the current driving speed, a level of the relaxed fatigue status may be further sub-classified. For example, when the difference between the current speed limit and the current driving speed is greater than a predetermined threshold value by 5 km/h, 10 km/h, and 15 km/h, the level of the relaxed fatigue status may be classified into the relaxed fatigue status of a first level, the relaxed fatigue status of a second level, and the relaxed fatigue status of a third level, respectively.

In one example, when the user takes off his outer clothing, when he/she continues to drive on a straight road with a high speed limit (i.e., a road where he/she may drive at a very high speed), when the measured heart rate is higher than the reference value, or when the volume of the call voice is greater than an average volume of the user's call voice, the user's fatigue status may be classified as the tense fatigue status among the negative emotion fatigues. For example, the average volume of the user's call voice described above may be calculated from the user's call record stored in the user device 300. Further, when the user's posture is different from a correct posture, when the seat massage function is used, or when the user frequently changes his/her sitting posture correctly, the user's fatigue status may be classified as the lack-of-exercise fatigue status.

Similarly, the fatigue type-related fatigue detection factors as shown in FIG. 3 are merely examples, and the present disclosure is not limited thereto. The fatigue status may be classified based on the fatigue type using various fatigue detection factors other than those as shown in FIG. 3.

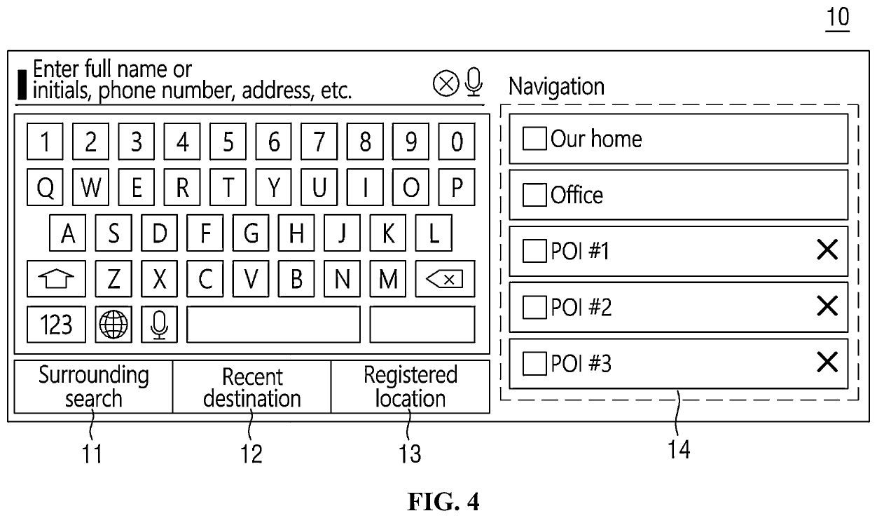

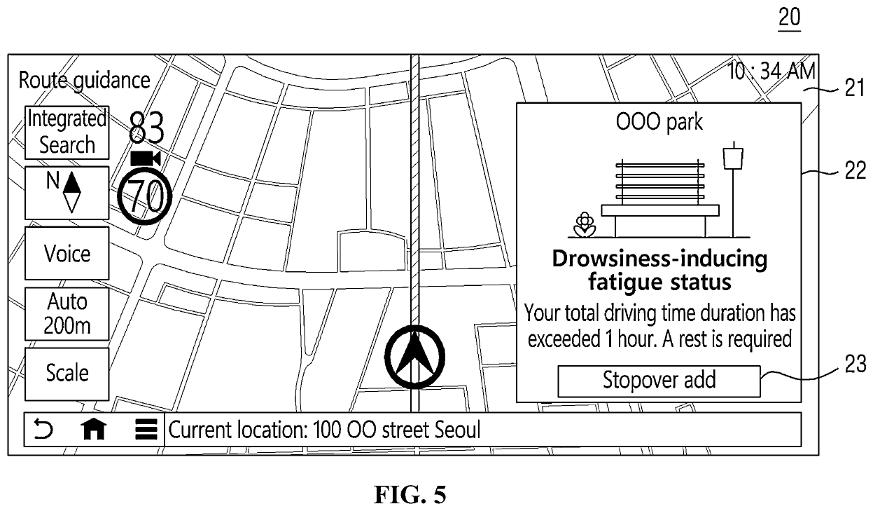

Hereinafter, referring to FIGS. 4 and 5, an user interface of the navigation system 100 is described.

FIG. 4 illustrates an example of an initial screen 10 of the navigation system 100 of FIG. 1. Referring to FIG. 4, the navigation system 100 may receive a search word from a user of the vehicle or the mobility apparatus 200, search for a route for one or more destinations corresponding to the input search word, guide the user along searched route, and display information related to the detected destination on the screen. Referring to FIG. 2, the initial screen 10 displays a surrounding search button 11, a recent destination button 12, and a registered location button 13. When the surrounding search button 11 is pressed, search categories of current location surrounding, destination surrounding, route surrounding, or local area surrounding may be displayed. When the recent destination button 12 is pressed, destinations corresponding to a recently entered search word may be displayed. When the registered location button 13 is pressed, destinations with a high search frequency (e.g., home, office, etc.) may be displayed. For example, locations 14 displayed at the right side of the initial screen 10 as shown in FIG. 2 may be a list of the registered destinations. The following describes a route guidance screen, and a corresponding stopover recommendation screen displayed after the user has entered the search word or selected a registered destination, with reference to FIG. 5.

FIG. 5 shows an example of a route guidance screen 20 of the navigation system 100 of FIG. 1. Referring to FIG. 5, the route guidance screen 20 according to an embodiment of the present disclosure displays a map 21, a stopover recommendation area 22, and a stopover add button 23. The map 21 displays the route being guided, and a current location and a driving direction along with traffic-related information. The stopover recommendation area 22 displays a stopover recommended for rest, and detailed information related to the stopover together. For example, in the example of FIG. 5, the user's total driving time duration has exceeded 1 hour, and thus the user's state is determined to be in the drowsiness-inducing fatigue status, and thus a park is recommended as the stopover with a message indicating that a rest is required. When the stopover add button 23 is pressed by the user, a route including the stopover may be determined as the final route, and route guidance may be newly started.

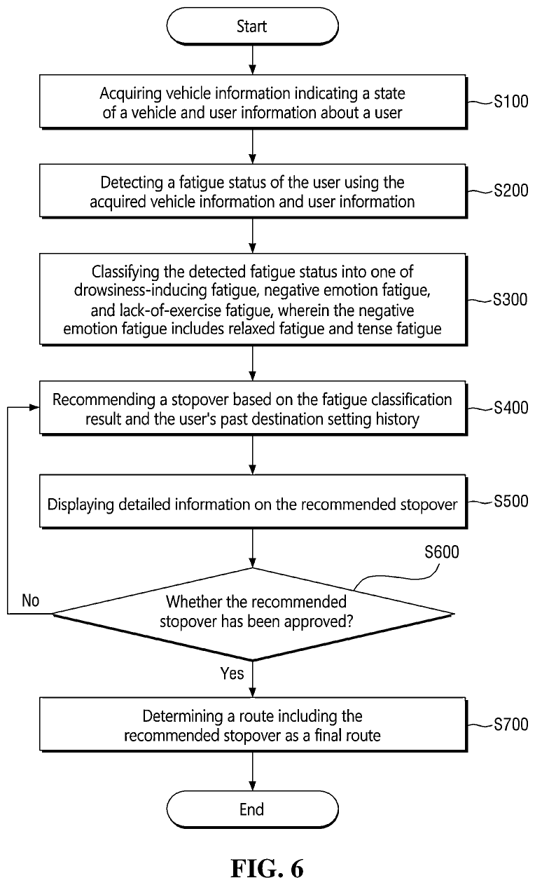

FIG. 6 is a flowchart for illustrating a stopover recommendation method based on a fatigue status according to an embodiment of the present disclosure. For reference, FIG. 6 shows the operations/steps performed in the navigation system 100 of FIG. 1. Therefore, in the descriptions as set forth below, it may be understood that when a subject of a specific step/operation is omitted, the step/operation is performed in the navigation system 100 of FIG. 1. Furthermore, as described with reference to FIG. 1, the mobility apparatus 200 of FIG. 1 is assumed to be the vehicle. However, the present disclosure is not limited thereto, and the embodiments as described below may be applied to all mobility apparatuses 200 other than vehicles. The following description is made with reference to FIG. 1 to FIG. 5 along with FIG. 6.

In operation S100, vehicle information indicating the state of the vehicle and user information may be acquired. As described with reference to FIG. 1, the vehicle information indicating the state of the vehicle 200 may include information on a location (GPS) of a vehicle, a driving speed, a total driving time duration, a window opening time or a frequency of window opening and closing, a brake pedal stepping strength, a signal indicating a departure of a preceding vehicle, information on media being played, a frequency of air conditioner control, whether ADAS (Advanced Driver Assistance Systems function) is used, whether a seat massage function is used, etc. which may be acquired from the vehicle. Furthermore, the user information may include the user's heart rate, a volume of voice during a call, the user's past destination setting history, whether or not he or she has visited a specific location, etc., which may be extracted from the navigation system 100 and the user device 300. Furthermore, the user information may further include the user's posture, a frequency of changing the posture correctly, a frequency of yawning, etc., which may be extracted from a camera installed in the vehicle 200. Furthermore, the user information may be extracted from the usage history of the navigation system 100 and the user device 300.

In operation S200, the user's fatigue status may be detected using the acquired vehicle information and user information. For example, the user's fatigue status may be detected when the total driving time duration has exceeded a threshold time duration, when yawning occurs frequently, when a difference between a speed limit and a current driving speed is gradually increasing (i.e., driving at an increasingly lower speed), when the vehicle does not start immediately despite a preceding vehicle movement-start notification signal, when the heart rate is low or high, when the air conditioner is frequently turned on or off, when the posture is constantly corrected, or when the ADAS function is used for an excessively long time, etc.

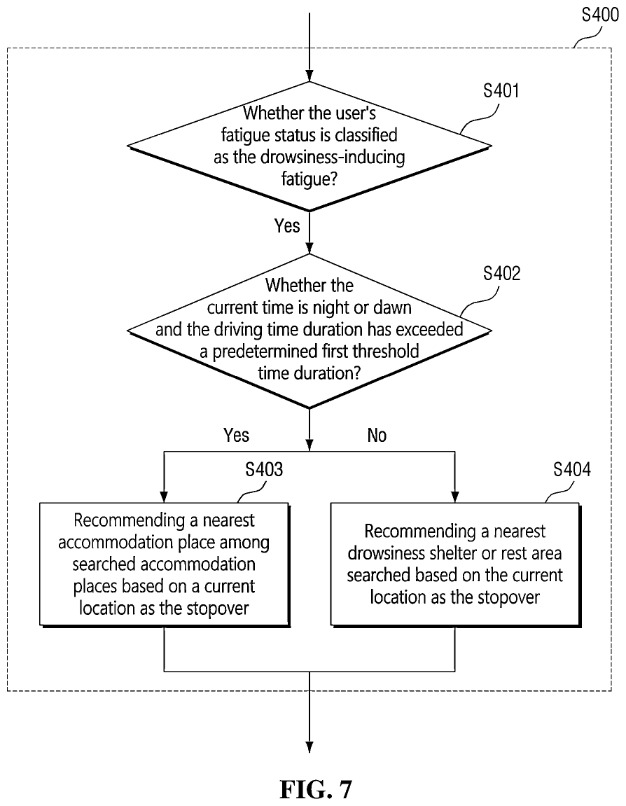

In operation S300, the detected fatigue status may be classified into one of the drowsiness-inducing fatigue, the negative emotion fatigue, and the lack-of-exercise fatigue. Among these fatigues, the negative emotion fatigue may be classified into the relaxed fatigue and the tense fatigue. For example, the classification of the fatigue type may be performed based on the fatigue detection factors shown in FIG. 3. Thereafter, in operation S400, a stopover may be recommended based on the fatigue classification result and past destination setting history. For example, from among the appropriate stopovers selected based on the fatigue classification result, a stopover that the user has visited may be recommended preferentially. Hereinafter, various embodiments of operation S400 are described with reference to FIGS. 7 to 9.

FIGS. 7 to 9 are flowcharts for illustrating embodiments of operation S400 of recommending a stopover based on the fatigue classification result of FIG. 6 and the past destination setting history.

Referring to FIG. 7, in operation S401, it may be determined whether the user's fatigue status is classified as the drowsiness-inducing fatigue. When the user's fatigue status is classified as the drowsiness-inducing fatigue (YES), in operation S402, it may be determined whether the current time is night or dawn and the driving time duration has exceeded a predetermined threshold time duration. When the current time is night or dawn and the total driving time duration has exceeded the predetermined threshold time duration (YES), in operation S403, the nearest accommodation place among the searched accommodation places based on the current location may be recommended as a stopover. In another example, when the current time is not night or dawn, or when the total driving time duration does not exceed the threshold time duration (NO), in operation S404, the nearest drowsiness shelter or rest area searched based on the current location may be recommended as a stopover. In this regard, the aforementioned threshold time duration may be determined based on the user's driving pattern acquired from the vehicle 200.

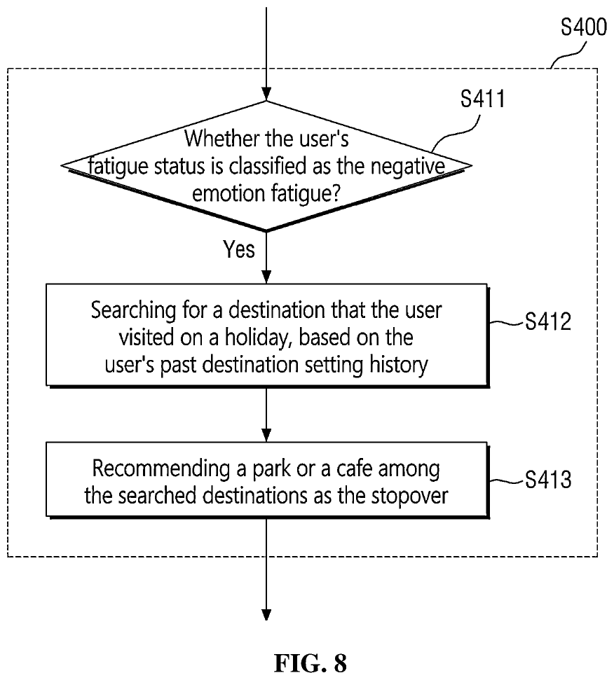

Referring to FIG. 8, in operation S411, it may be determined whether the user's fatigue status is classified as the negative emotion fatigue. When the user's fatigue status is classified as the negative emotion fatigue (YES), in operation S412, a destination that the user visited on a holiday may be searched for based on the user's past destination setting history. Thereafter, in operation S413, a park or a cafe among the searched destinations may be recommended as a stopover. In one example, depending on whether the negative emotion fatigue is classified as the relaxed fatigue or the tense fatigue, places of other types other than parks or cafes may also be recommended as stopovers.

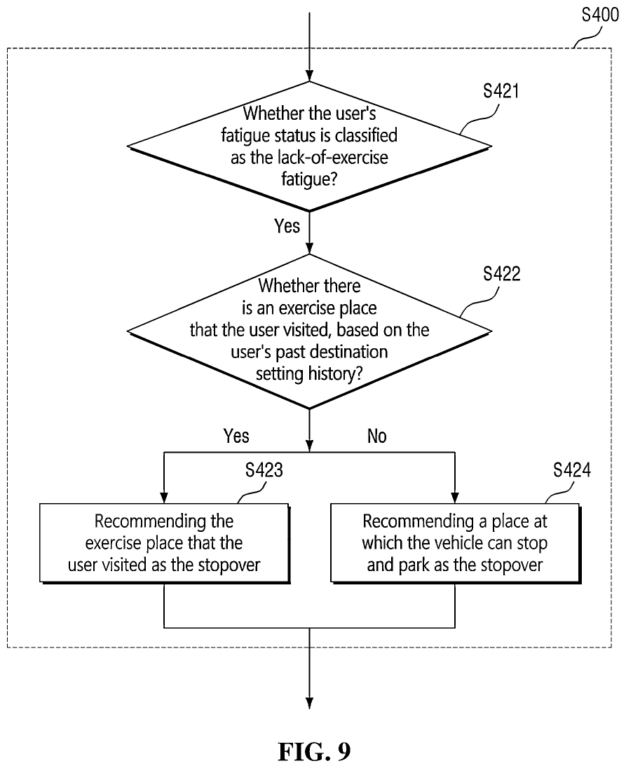

Referring to FIG. 9, in operation S421, it may be determined whether the user's fatigue status is classified as the lack-of-exercise fatigue. When the user's fatigue status is classified as the lack-of-exercise fatigue (YES), in operation S422, it may be determined whether there is an exercise place that the user visited. When there is the exercise place that the user visited (YES), in operation S423, the exercise place that the user visited may be recommended as a stopover. In another example, when there is no exercise place that the user visited (NO), a place at which the vehicle stops and parks may be recommended as a stopover.

Returning to FIG. 6, in operation S500, detailed information about the recommended stopover may be displayed. For example, the detailed information may include the user's fatigue status based on which the stopover is recommended, a type of rest that may be taken in the stopover, and an expected driving time to drive along a route including the recommended stopover thereto. In operation S600, it may be determined whether the user approves the recommended stopover. For example, the user may check the recommended stopover and then press the add stopover button on the screen as shown in FIG. 5 to approve the recommended stopover. When the recommended stopover has been approved by the user (YES), in operation S700, a route including the recommended stopover may be determined as the final route in response to the user's approval. In one example, when the recommended stopover is not approved by the user (NO), the process returns to operation S400, in which a new stopover may be recommended as the stopover.

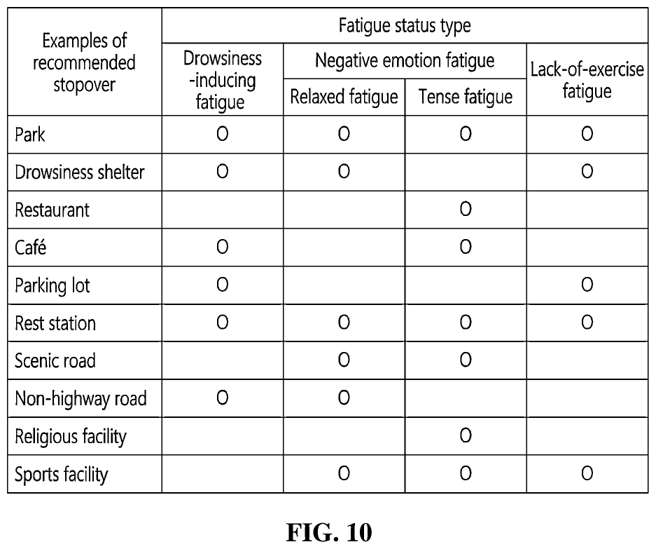

FIG. 10 illustrates an example of a stopover that may be recommended based on the fatigue type according to an embodiment of the present disclosure. Referring to FIG. 10, when the user is in the drowsiness-inducing fatigue status, parks, drowsiness shelters, cafes, parking lots, rest areas, and national parks may be recommended as the stopover. When the user is in the relaxed fatigue status, parks, drowsiness shelters, rest areas, scenic roads, non-highway roads, and sports facilities may be recommended as the stopover. When the user is in the tense fatigue status, parks, restaurants, cafes, rest areas, scenic roads, religious facilities, and sports facilities may be recommended as the stopover. When the user is in the lack-of-exercise fatigue status, parks, drowsiness shelters, parking lots, rest areas, and sports facilities may be recommended as the stopover. The fatigue type-related stopover shown in FIG. 10 is an example, and the present disclosure is not limited thereto, and other places may be recommended as the stopover depending on the user's past destination visit history.

FIG. 11 is a block diagram showing the hardware configuration of a computing device 500 for performing the stopover recommendation method based on the fatigue status according to an embodiment of the present disclosure.

Referring to FIG. 11, the computing device 500 may include one or more processors 510, a bus 530, a communication interface 540, a memory 520 that loads a computer program executed by the processor 510 therein, and a storage 550 that stores a computer program 560 therein. However, only components related to an embodiment of the present disclosure are illustrated in FIG. 11. Therefore, a person skilled in the art in the technical field to which the present disclosure belongs may understand that other general components than the components as illustrated in FIG. 11 may be included in the computing device.

In other words, the computing device 500 may include various components in addition to the components as illustrated in FIG. 11. Furthermore, in some cases, the computing device 500 may be configured such that some of the components as illustrated in FIG. 11 are omitted. Hereinafter, each of the components of the computing device 500 is described.

The processor 510 may control all operations of the components of the computing device 500. The processor 510 may be configured to include at least one of a CPU (Central Processing Unit), an MPU (Micro Processor Unit), an MCU (Micro Controller Unit), a GPU (Graphics Processing Unit), or any further type of a processor well known in the technical field of the present disclosure. Furthermore, the processor 510 may perform computations of at least one application or program for executing operations/methods according to some embodiments of the present disclosure. The computing device 500 may have one or more processors.

Next, the memory 520 may store therein various data, commands, and/or information. The memory 520 may load therein the computer program 560 from the storage 550 to execute operations/methods according to some embodiments of the present disclosure. The memory 520 may be embodied as a volatile memory such as RAM. However, the present disclosure is not limited thereto.

Next, the bus 530 may provide a communication function between the components of the computing device 500. The bus 530 may be embodied as various types of buses such as an address bus, a data bus, and a control bus.

Next, the communication interface 540 may support wired and wireless Internet communication of the computing device 500. Furthermore, the communication interface 540 may support various communication schemes other than Internet communication. To this end, the communication interface 540 may be configured to include a communication module well known in the technical field of the present disclosure.

Next, the storage 550 may non-temporarily store therein one or more computer programs 560. The storage 550 may be configured to include a non-volatile memory such as Read Only Memory (ROM), Erasable Programmable ROM (EPROM), Electrically Erasable Programmable ROM (EEPROM), flash memory, a hard disk, a removable disk, or any form of computer-readable recording medium well known in the art to which the present disclosure belongs.

Next, the computer program 560 may include one or more instructions that cause the processor 510 to perform the operations/methods according to various embodiments of the present disclosure when being loaded into the memory 520. That is, the processor 510 may execute one or more loaded instructions to perform the operations/methods according to various embodiments of the present disclosure.

For example, the computer program 560 may include instructions for acquiring vehicle information indicating a state of a vehicle and user information about a user, detecting a fatigue status of the user using the acquired vehicle information and user information, classifying the detected fatigue status into one of drowsiness-inducing fatigue, negative emotion fatigue, and lack-of-exercise fatigue, wherein the negative emotion fatigue includes relaxed fatigue and tense fatigue, recommending a stopover based on the fatigue classification result and the user's past destination setting history, displaying detailed information on the recommended stopover, and determining a route including the recommended stopover as a final route, in response to the user's approval with the recommended stopover.

According to an embodiment of the present disclosure, the user's fatigue status is detected, and a stopover for rest is recommended according to the fatigue type of the detected fatigue status, thereby providing the user with a safer and more positive driving experience. This may prevent accidents that may occur due to careless driving. The system installed in the vehicle checks the user's state and provides functions to assist the driving such that the user's trust in the vehicle software may also increase.

Various embodiments and the effects thereof according to the present disclosure have been mentioned with reference to FIGS. 1 through 11. The effects according to the technical spirit of the present disclosure are not limited to those mentioned above, and other effects not mentioned will be clearly understood by one of ordinary skill in the art from the description below.

While all components comprising the embodiments of the present disclosure have been described as being combined or operating in conjunction, it should not be understood that the present disclosure is limited to such embodiments. That is, within the scope of the objectives of the present disclosure, all such components can selectively be combined and operate in one or more configurations.

Although operations are illustrated in a specific order in the drawings, it should not be understood that the operations must be performed in that specific order or sequentially, or that all the illustrated operations are required to achieve desired results. In certain circumstances, multitasking and parallel processing may be advantageous. Furthermore, the separation of various components in the described embodiments should not be understood as necessary, and the described program components and systems can generally be integrated into a single software product or packaged into multiple software products.

While the embodiments of the present disclosure have been described with reference to the attached drawings, it will be understood by one skilled in the art that the present disclosure can be implemented in other specific forms without departing from the technical spirit or essential characteristics thereof. Therefore, the described embodiments should be considered in all respects as illustrative and not restrictive. The scope of the present disclosure is to be interpreted by the following claims, and all technical spirits within the equivalent scope are to be interpreted as included within the rights of the present disclosure.

What is claimed is:

1. A stopover recommendation method performed by a computing device, the method comprising:
acquiring vehicle information indicating a state of a vehicle and user information about a user;
detecting a fatigue status of the user using the vehicle information and the user information;
classifying the fatigue status into a drowsiness-inducing fatigue, a negative emotion fatigue, or a lack-of-exercise fatigue, wherein the negative emotion fatigue includes a relaxed fatigue and a tense fatigue;
recommending a stopover based on the fatigue status of the user and a past destination setting history of the user;
displaying detailed information on the recommended stopover; and
determining a route including the recommended stopover as a final route in response to an approval of the user on the recommended stopover.

2. The stopover recommendation method of claim 1, wherein the vehicle information includes a location (GPS) of a vehicle, a driving speed, a total driving time duration, a window opening time, a frequency of window opening and closing, a brake pedal stepping strength, a signal indicating a departure of a preceding vehicle, information on media being played, a frequency of air conditioner control, whether ADAS (Advanced Driver Assistance Systems function) is used, or whether a seat massage function is used,
wherein the user information is extracted from usage history of the computing device and a user device of the user, and
wherein the user information includes a heart rate of the user, a volume of a voice of the user during a call, the past destination setting history of the user, whether or not the user has visited a specific location, or a posture of the user.

3. The stopover recommendation method of claim 2, wherein the classifying of the fatigue status includes:
when a driving timing is evening, night, or dawn, when the total driving time duration is equal to or greater than a first threshold time duration, or when a window opening time duration is equal to or greater than a second threshold time duration,
classifying the fatigue status as the drowsiness-inducing fatigue,
wherein each of the first threshold time duration and the second threshold time duration is determined based on a driving pattern of the user acquired from the vehicle.

4. The stopover recommendation method of claim 2, wherein the classifying of the fatigue status includes:
when the heart rate is smaller than a reference value, when a brake pedal stepping strength is smaller than an average brake pedal stepping strength of the user, when a difference between a speed limit on a road on which the vehicle is currently driving and a current driving speed of the vehicle is larger than a predetermined threshold value, or when the vehicle does not start despite a preceding vehicle movement-start notification signal,
classifying the fatigue status as the relaxed fatigue of the negative emotion fatigue,
wherein the brake pedal stepping strength is stored in the vehicle every time the user drives the vehicle,
wherein the average brake pedal stepping strength is calculated from values of the brake pedal stepping strength stored in the vehicle, and
wherein the predetermined threshold value is determined as an average of difference in values between a driving speed stored in a navigation system of the vehicle and the speed limit on the road on which the vehicle is currently driving.

5. The stopover recommendation method of claim 2, wherein the classifying of the fatigue status includes:
when the heart rate is higher than a reference value, or when the volume of the voice of the user during the call is greater than an average volume of the voice of the user during the call,
classifying the fatigue status as the tense fatigue of the negative emotion fatigue,
wherein the average volume of the voice of the user during the call is calculated from a call record of the user stored in the user device.

6. The stopover recommendation method of claim 2, wherein the classifying of the fatigue status includes:
when the posture of the user is different from a correct posture, or when the seat massage function is activated,
classifying the fatigue status as the lack-of-exercise fatigue.

7. The stopover recommendation method of claim 1, wherein the recommending of the stopover includes:
determining whether the fatigue status of the user is classified as the drowsiness-inducing fatigue,
when the fatigue status of the user is classified as the drowsiness-inducing fatigue, determining whether a current time is night or dawn and a total driving time duration has exceeded a first threshold time duration,
when the current time is night or dawn and the total driving time duration has exceeded the first threshold time duration, recommending a nearest accommodation place among searched accommodation places based on a current location as the recommended stopover, and
when the current time is not night or dawn, or when the total driving time duration has not exceeded the first threshold time duration, recommending a nearest drowsiness shelter or a nearest rest area searched based on the current location as the recommended stopover,
wherein the first threshold time duration is determined based on a driving pattern of the user acquired from the vehicle.

8. The stopover recommendation method of claim 1, wherein the recommending of the stopover includes:
determining whether the fatigue status of the user is classified as the negative emotion fatigue,
when the fatigue status of the user is classified as the negative emotion fatigue, searching for destinations that the user visited on a holiday based on the past destination setting history of the user; and
recommending a park or a cafe among the destinations as the recommended stopover.

9. The stopover recommendation method of claim 1, wherein the recommending of the stopover includes:
determining whether the fatigue status of the user is classified as the lack-of-exercise fatigue;
when the fatigue status of the user is classified as the lack-of-exercise fatigue, determining whether there is an exercise place that the user visited, based on the past destination setting history of the user; and
when there is the exercise place that the user visited, recommending the exercise place that the user visited as the recommended stopover; and
when there is no exercise place that the user visited, recommending a place at which the vehicle can stop and park as the recommended stopover.

10. The stopover recommendation method of claim 1, wherein the detailed information on the recommended stopover includes the fatigue status of the user, a type of rest that may be taken at the recommended stopover, or an expected driving time to drive along a route including the recommended stopover thereto.

11. A computing device comprising:
a processor; and
a memory connected to the process and configured to store therein instructions,
wherein when the instructions are executed by the processor, the instructions cause the processor to perform a method comprising:
acquiring vehicle information indicating a state of a vehicle and user information about a user;
detecting a fatigue status of the user using the vehicle information and the user information;
classifying the fatigue status into a drowsiness-inducing fatigue, a negative emotion fatigue, or a lack-of-exercise fatigue, wherein the negative emotion fatigue includes a relaxed fatigue and a tense fatigue;
recommending a stopover based on the fatigue status of the user and a past destination setting history of the user;
displaying detailed information on the recommended stopover; and
determining a route including the recommended stopover as a final route in response to an approval of the user on the recommended stopover.

12. The computing device of claim 11, wherein the vehicle information includes a location (GPS) of a vehicle, a driving speed, a total driving time duration, a window opening time, a frequency of window opening and closing, a brake pedal stepping strength, a signal indicating a departure of a preceding vehicle, information on media being played, a frequency of air conditioner control, whether ADAS (Advanced Driver Assistance Systems function) is used, or whether a seat massage function is used,
wherein the user information is extracted from usage history of the computing device and a user device of the user, and
wherein the user information includes a heart rate of the user, a volume of a voice of the user during a call, the past destination setting history of the user, whether or not the user has visited a specific location, or a posture of the user.

13. The computing device of claim 12, wherein the classifying of the fatigue status includes:
when a driving timing is evening, night, or dawn, when the total driving time duration is equal to or greater than a first threshold time duration, or when a window opening time duration is equal to or greater than a second threshold time duration,
classifying the fatigue status as the drowsiness-inducing fatigue,
wherein each of the first threshold time duration and the second threshold time duration is determined based on a driving pattern of the user acquired from the vehicle.

14. The computing device of claim 12, wherein the classifying of the fatigue status includes:
when the heart rate is smaller than a reference value, when a brake pedal stepping strength is smaller than an average brake pedal stepping strength of the user, when a difference between a speed limit on a road on which the vehicle is currently driving and a current driving speed of the vehicle is larger than a predetermined threshold value, or when the vehicle does not start despite a preceding vehicle movement-start notification signal,
classifying the fatigue status as the relaxed fatigue of the negative emotion fatigue,
wherein the brake pedal stepping strength is stored in the vehicle every time the user drives the vehicle,
wherein the average brake pedal stepping strength is calculated from values of the brake pedal stepping strength stored in the vehicle, and
wherein the predetermined threshold value is determined as an average of difference in values between a driving speed stored in a navigation system of the vehicle and the speed limit on the road on which the vehicle is currently driving.

15. The computing device of claim 12, wherein the classifying of the fatigue status includes:
when the heart rate is higher than a reference value, or when the volume of the voice of the user during the call is greater than an average volume of the voice of the user during the call,
classifying the fatigue status as the tense fatigue of the negative emotion fatigue,
wherein the average volume of the voice of the use during the call is calculated from a call record of the user stored in the user device.

16. The computing device of claim 12, wherein the classifying of the fatigue status includes:
when the posture of the user is different from a correct posture, or when the seat massage function is activated,
classifying the fatigue status as the lack-of-exercise fatigue.

17. The computing device of claim 11, wherein the recommending of the stopover includes:
determining whether the fatigue status of the user is classified as the drowsiness-inducing fatigue,
when the fatigue status of the user is classified as the drowsiness-inducing fatigue, determining whether a current time is night or dawn and a total driving time duration has exceeded a first threshold time duration,
when the current time is night or dawn and the total driving time duration has exceeded the first threshold time duration, recommending a nearest accommodation place among searched accommodation places based on a current location as the recommended stopover, and
when the current time is not night or dawn, or when the total driving time duration has not exceeded the first threshold time duration, recommending a nearest drowsiness shelter or a nearest rest area searched based on the current location as the recommended stopover,
wherein the first threshold time duration is determined based on a driving pattern of the user acquired from the vehicle.

18. The computing device of claim 11, wherein the recommending of the stopover includes:
determining whether the fatigue status of the user is classified as the negative emotion fatigue,
when the fatigue status of the user is classified as the negative emotion fatigue, searching for destinations that the user visited on a holiday based on the past destination setting history of the user; and
recommending a park or a cafe among the destinations as the stopover.

19. The computing device of claim 11, wherein the recommending of the stopover includes:
determining whether the fatigue status of the user is classified as the lack-of-exercise fatigue;
when the fatigue status of the user is classified as the lack-of-exercise fatigue, determining whether there is an exercise place that the user visited, based on the past destination setting history of the user; and
when there is the exercise place that the user visited, recommending the exercise place that the user visited as the recommended stopover; and
when there is no exercise place that the user visited, recommending a place at which the vehicle can stop and park as the recommended stopover.

20. The computing device of claim 11, wherein the detailed information on the recommended stopover includes the fatigue status of the user, a type of rest that may be taken at the stopover, or an expected driving time to drive along a route including the recommended stopover thereto.
